(19) United States
(12) Patent Application Publication
(10) Pub. No.: US 2006/0235290 A1
(43) Pub. Date: Oct. 19, 2006

(54) Method and apparatus for positioning a cutting tool for orthopedic surgery using a localization system

(75) Inventors: Christian Gabriel, Tuttlingen (DE); Francois Leitner, Uriage (FR); Richard Schill, Eaulogne (FR)

Correspondence Address: Theodore Naccarella, Esquire;Synnestvedt & Lechner LLP, 2600 Aramark Tower, 1101 Market Street, Philadelphia, PA, 19107-2950, US

(73) Assignee: Aesculap AG & CO. KG, Tuttlingen (DE)

(21) Appl. No.: 11/338,450

(22) Filed: Jan. 24, 2006

Related U.S. Application Data
(60) Provisional application No. 60/668,048, filed on Apr. 4, 2005.

(51) Int. Cl. A61B 5/05

(52) U.S. Cl. 600/407

(57) ABSTRACT

The present invention provides methods and apparatus that permit one to position a cutting jig or other component using a localization device to navigate different degrees of freedom of the component in discrete, sequential steps. For instance, a first mounting pin for a cutting jig that sets one or more, but fewer than all, degrees of freedom of the cutting jig is navigated into position using the localization device. For instance, the first mounting pin might set the height and the slope of the cutting plane of the jig. Next, a marker is mounted on the cutting jig and the cutting jig is slid onto the mounted pin. Then the cutting block is navigated in another degree of freedom, for instance, to set the varus-valgus angle of the cutting plane by rotating the jig about the axis of the pin. A second mounting pin for the jig is affixed to the bone based on that navigation.

### CROSS REFERENCE TO RELATED APPLICATION

This application claims priority under 35 U.S.C. §119(e) to U.S. provisional patent application No. 60/668,048 filed Apr. 4, 2005, which is fully incorporated herein by reference.

### FIELD OF THE INVENTION

The present invention relates to surgical navigation systems, sometimes called localization devices. More particularly, the present invention relates to methods and apparatus for positioning a cutting tool for orthopedic surgery using a surgical navigation system.

### BACKGROUND OF THE INVENTION

In an exemplary surgical navigation system 100 such as illustrated in FIG. 1, at least two sensors 114a, 114b (e.g., infrared cameras) mounted in a housing 128 are used to detect a plurality of markers 116a, 116b, 116c, 116d, 116e that can be mounted on the patient's bones 105a, 105b and/or on surgical tools 124. More particularly, the cameras 114a, 114b are coupled to a computer 112 that analyzes the images obtained by the cameras and detects the positions and orientations of the various bones and/or tools bearing the markers during the surgery and calculates and displays useful information for performing the surgery to the surgeon on a monitor 122. The computer system may be provided in a portable cart 108 and may include a memory 110 for storing data, a keyboard 120, and/or foot pedals 118 for entering data.

Typically two or more of the markers 116a-116e are used simultaneously. One such surgical navigation system is the OrthoPilot available from Aesculap, Inc. of Center Valley, Pa., USA.

The following discussion will use the OrthoPilot as an exemplary surgical navigation system, but it should be understood that the OrthoPilot is merely exemplary of a typical surgical navigation system.

With reference to FIG. 2A, which is an enlarged view of an exemplary marker 116 mounted on a sagittal saw 202, each marker 116 comprises a base with a mounting mechanism 217 on one end for mounting to a complementary mounting mechanism 201 on a piece of medical equipment such as sagittal saw 202 of FIG. 2A, surgical pointer 124 of FIG. 1, a bone screw, or a cutting jig. Extending from the other end of the base are at least three infrared LED transmitters 208. Alternately, instead of transmitters, the system could utilize markers 116a bearing infrared reflectors 208a, as shown in FIG. 2B, which illustrates an exemplary marker 116a of the reflector type. When using reflectors, the surgical navigation system includes an infrared light source 107 (FIG. 1) directed towards the surgical field so that the reflectors 208 will reflect infrared light back to the two cameras 114a, 114b. With at least two cameras and at least three transmitters 208 (or reflectors 208a) per marker, sufficient information is available to the computer to determine the exact position and orientation of each marker 116 (or 116a) in all six degrees of freedom.

In most surgical navigation procedures, it is necessary to discern the markers 116 or 116a from each other. This can be done in several different ways. If LED transmitters are used, each transmitter 208 can be timed to emit light only during a specific time interval that the computer knows is the time interval assigned to that particular transmitter on that particular marker. The LEDs are illuminated in sequence at a very high rate so that the computer has virtually continuous information as to the exact location of every LED. Alternately, when using reflectors, each marker 116a may have its three or more reflectors 208a positioned in slightly different relative positions to each other so that the computer can discern which marker it is observing by determining the geometric relationship between the three or more reflectors 208a on the marker 116a.

Referring back to FIG. 1, the markers 116 are fixedly mounted on bones 105 (via bone screws) and or medical instruments 124 (FIG. 1) or 202 (FIG. 2A) positioned within the field of view of the cameras 114a, 114b so that the computer 112 can track the location and orientation of those bones and/or medical instruments. The computer will then generate useful information to help the surgeon determine appropriate locations or alignments for prosthetic implants, cutting jigs, and the like and display it in a display 123 on the monitor 122.

The mounting mechanism at the end of the base of the marker is designed to mate with a complementary mounting mechanism on the surgical instrument in only one position and orientation. The computer is preprogrammed with information relating to the position of the operational portion of the medical instrument relative to the position of the marker when mounted on it. In this manner, by detecting the position and orientation of the marker, the computer will also know the position and orientation of the medical instrument and its operational portion. For instance, the medical instrument may be the pointer 124 shown in FIG. 1 having a tip 124a, the exact position of which is known relative to the marker 116a.

One known use for surgical navigation systems is in knee replacement surgery. In Total Knee Arthroplasty (TKA) surgery, for instance, the patient's knee joint 136 is replaced with prosthetic components including a prosthetic tibial component and a prosthetic femoral component. In order to mount the prosthetic components, the bottom of the patient's femur 105b and the top of the tibia 105a must be removed (see FIG. 1). This is done by cutting off the ends of those bones using a surgical saw such as sagittal saw 202 shown in FIG. 2A. The various bone cuts must be made precisely because the prosthetic components are designed to mount to the bones in a specific way. For instance, in TKA, at least some of the cut bone surfaces must be precisely aligned relative to the mechanical axis of the patient's leg (commonly exactly perpendicular to the mechanical axis). The navigation system can be used to track markers mounted to the femur and tibia and determine and track the mechanical axis of the patient's bones relative to markers as the leg is moved. Then, another marker can be mounted to a cutting jig for cutting the femur or tibia and the navigation system can be used to display the position of the cutting jig relative to the mechanical axis of the bone (which is still being tracked via the marker mounted on the bone) so that the surgeon can determine when the jig is positioned in exactly the desired orientation relative to the bone for making the cut. The surgeon can then affix the jig to the bone in that position and make the cut.

The surgeon must accurately position the cutting jig in at least three degrees of freedom. Particularly, the height, anterior/posterior slope (commonly and hereinafter referred to simply as slope), and varus-valgus angle of the cutting plane must be set very precisely relative to the mechanical axis of the bone. In the exemplary OrthoPilot surgical navigation system, to cut the tibia, the system shows on the computer monitor the orientation of the cutting plane of the cutting jig relative to the mechanical axis of the tibia in two planar views, namely, the frontal view (in which the varus-valgus angle of the cutting plane is visible), and the lateral (or sagittal) view (in which the slope of the cutting plane is visible). It also shows the height of the cutting plane relative to the tibial plateau in at least one of the two views. The surgeon must manipulate the jig until it is perpendicular to the mechanical axis of the bone in at least two degrees of freedom (varus-valgus and slope) and the displayed height is the desired height for the cut (the third degree of freedom). The surgeon then must attach the cutting jig to the tibia in this position and saw the top of the tibia off using the cutting jig. A similar process is repeated for the femur using a suitable femoral cutting jig.

Some surgeons find it difficult to position a jig accurately using surgical navigation systems because they must precisely position the cutting jig on the bone in multiple degrees of freedom while trying to looking at both the computer monitor and the patient's knee, and then mount the jig to the bone with two or more pins using a power tool while not moving the jig.

It is an object of the present invention to provide an improved method and apparatus for surgical navigation.

It is another object of the present invention to provide an improved method and apparatus for mounting a cutting jig using a surgical navigation system.

It is a further object of the present invention to provide an improved method and apparatus for positioning two components relative to each other using a localization system.

### SUMMARY OF THE INVENTION

The present invention provides methods and apparatus that overcome the aforementioned problems by permitting one to position a cutting jig or other component using a localization device to navigate different degrees of freedom of the component in discrete, sequential steps. In one embodiment for mounting a cutting jig, for instance, a first mounting pin for the cutting jig that sets one or more, but fewer than all, degrees of freedom of the cutting jig is navigated into position using the localization device. For instance, the first mounting pin (navigated) might set the height and the slope of the cutting plane of the jig. Next, a marker is mounted on the cutting jig and the cutting jig is slid onto the mounted pin. Then the cutting block is navigated in another degree of freedom, for instance, to set the varus-valgus angle of the cutting plane by rotating the jig about the axis of the pin. A second mounting pin for the jig is affixed to the bone based on that navigation.

In other embodiments, the second pin may be navigated separately such that there is no navigation of the cutting jig itself, but just of the two pins. In other embodiments, each degree of freedom may be navigated in a discrete step.

### BRIEF DESCRIPTION OF THE DRAWINGS

FIG. 1 is an illustration of a surgical navigation system being used for total knee arthroplasty surgery in accordance with the prior art.

FIG. 2A is a close-up perspective view of a marker of the LED emitter type for use with a surgical navigation system mounted on a surgical sagittal saw in accordance with the prior art.

FIG. 2B is a perspective view of a marker of the reflection type.

FIG. 3 is an illustration of a display screen for navigating a tibial cutting jig in accordance with the prior art.

FIG. 4 is a perspective view of a knee joint with an exemplary tibial cutting jig bearing a marker mounted thereon.

FIG. 5 is a perspective view of a surgical pin bearing a marker in accordance with the present invention.

FIG. 6 is an illustration of a display screen for a first step of navigating a tibial cutting jig in accordance with the present invention.

FIG. 7 is an illustration of a display screen for a second step of navigating a tibial cutting jig in accordance with the present invention.

FIG. 8 is a diagram illustrating the varus-valgus angle and slope relative to the mechanical axis of the leg.

FIG. 9 is a diagram illustrating calculation for determining height of the cutting plane.

FIG. 10 is a diagram illustrating calculations for determining the slope of the cutting plane.

### DETAILED DESCRIPTION OF THE INVENTION

A description of a suitable localization device for use in connection with the present invention is found in U.S. Pat. No. 6,385,475 to Cinquin et al., incorporated herein by reference.

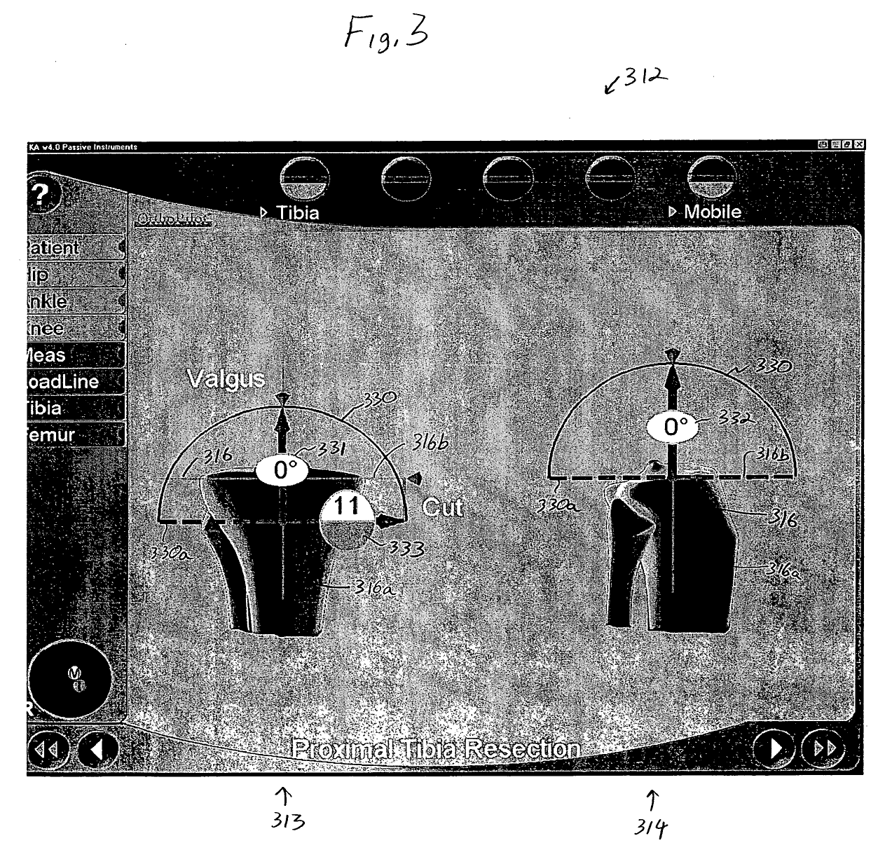

In order to assist the surgeon in this task, a surgical navigation system, for example, may display a screen such as the screen 312 shown in FIG. 3 which provides both a frontal view 313 and a lateral (also called sagittal) view 314 of the tibia and the cutting jig. In both views, a green cross-hair 316 (green being represented by the lighter lines) represents the mechanical axis of the tibia, with the vertical line 316a representing the mechanical axis of the tibia and the horizontal line 316b representing the plane perpendicular to that mechanical axis. The horizontal line 316b also represents the height of the tibial plateau in the frontal view 313. In the lateral view 314, the position of the line 316b is irrelevant. A red semicircle 330 (red being represented by the heavier lines in the Figure) in both views of the display represents the cutting jig. The display shows the relative position of the cutting jig to the mechanical axis of the tibia by showing the position of the red semicircle 330 relative to the green cross-hair 316. The display may also redundantly display the same information by showing (1) the numerical difference in angle between the jig and the mechanical axis in the frontal plane, as seen in circle 331, (2) the numerical difference in angle between the jig and the mechanical axis in the lateral plane, as seen in circle 332, and (3) the difference in height between the cutting jig and the tibial plateau in millimeters, as seen in circle 333.

While each patient is different, for sake of simplicity, let us assume that the proper position of the cutting jig is achieved when the cutting plane is perpendicular to the mechanical axis of the tibia (in both the frontal and lateral planes) and is equal 11 mm below the height to the tibial plateau. This orientation exists when the base 330a of the red semicircle 330 is parallel to the line 316b of the green cross-hair 316 in both the frontal and lateral views and the base 330a of the red semicircle overlaps line 316b in the lateral plane, i.e., zero degrees difference in the frontal plane (varus-valgus angle), zero degrees difference in the lateral plane (slope). This position/height exists when the circle 609 shows the number 11.

In order to position the cutting jig, the surgeon must manually hold the position of the jig in the aforementioned three degrees of freedom while directing his attention to both the computer monitor and the surgical field and mounting at least two pins into the bone through mounting holes in the jig using a power tool to affix the jig to the bone without moving the jig while doing so. This requires substantial concentration and manual dexterity.

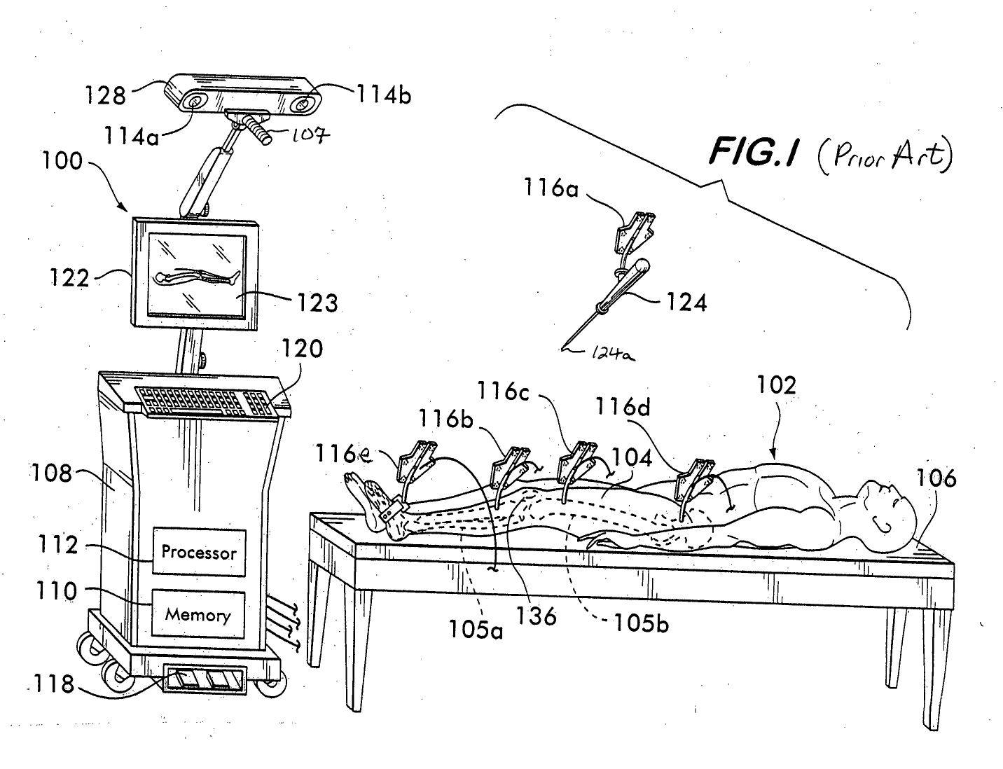

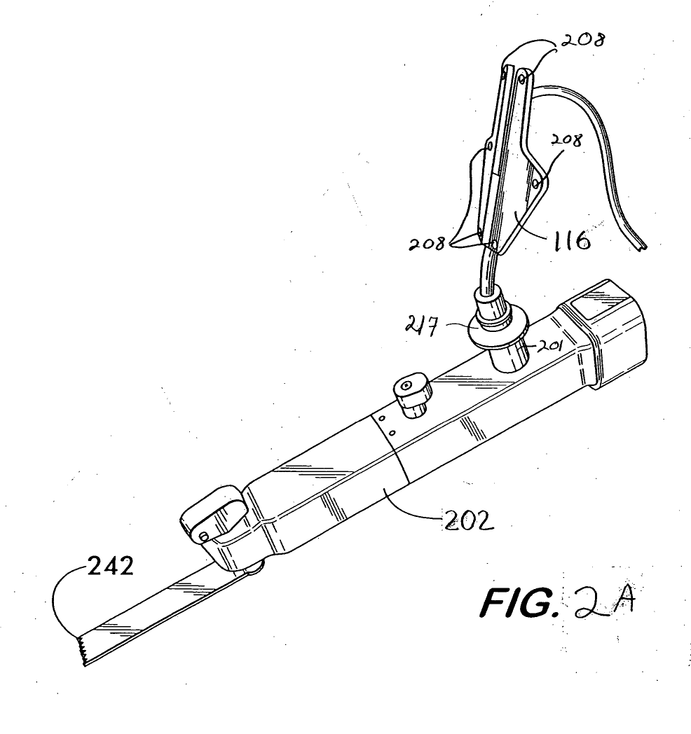

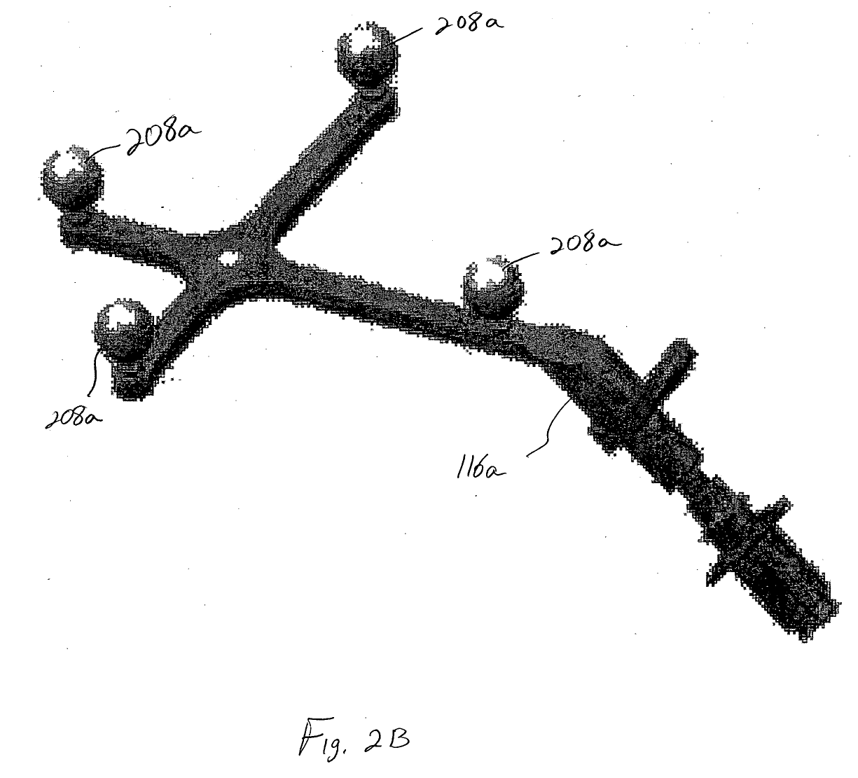

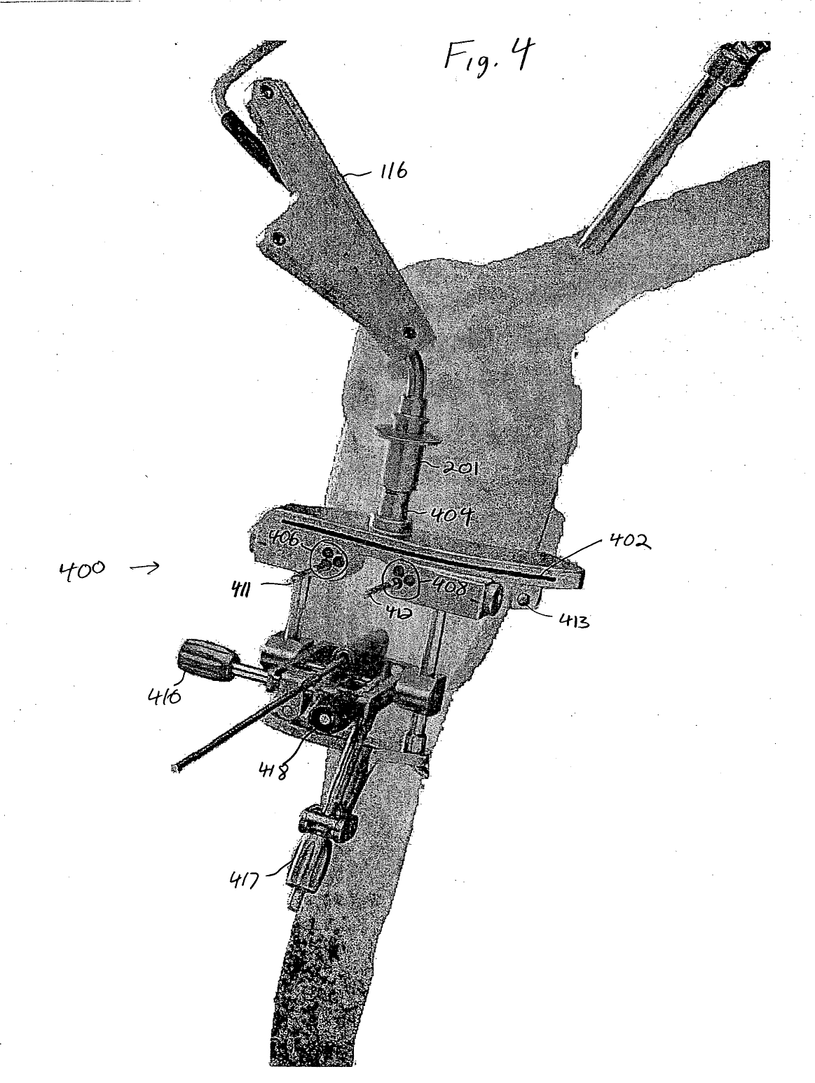

FIG. 4 is a perspective view of an exemplary cutting jig that can be navigated using a surgical navigation system. In particular, FIG. 4 depicts the tibial cutting jig 400 supplied with the OrthoPilot surgical navigation system sold by Aesculap, Inc. of Center Valley, Pa. USA. The jig 400 comprises a slot 402 within which the saw blade 242 of a surgical saw 202 (FIG. 2A) can be inserted to cut a tibia in order for it to accept a tibial prosthesis. The slot 402 is only slightly wider than the saw blade such that, when the tibial cutting jig 400 is properly mounted on the tibia, the saw blade can be inserted into it to make a very precise, controlled cut of the tibia. The cutting jig 400 is supplied with a mounting mechanism 404 for accepting the complementary mounting mechanism 201 of a marker, such as the markers 116 discussed above in connection with FIG. 1. The marker 116 can be mounted on the cutting jig 400 in only one precise orientation.

As previously described, the computer software associated with this surgical navigation system is preprogrammed to know the position of the cutting plane of the cutting jig relative to the position of the marker.

The cutting jig 400 further comprises two sets of three through holes 406 and 408 that are used for mounting the cutting jig to the tibia. Particularly, one hole from each set is selected to be slid over one of pins 411, 412 that are rigidly attached to the tibia.

One practice for mounting a tibial cutting jig to the tibia is to mount the marker 116 to the cutting jig 400 and then navigate the cutting jig in three degrees of freedom, namely, the slope, the varus-valgus angle, and height relative to the tracked tibia. When the monitor display of the cutting jig shows that the jig is in the proper orientation in the aforementioned three degrees of freedom, the surgeon would then attach a mounting pin to a drill and drill the pin into the tibia through one of the holes in one of hole sets 406, 408 of the cutting jig. This is then repeated for a second pin using one of the holes on the other of the two hole sets 406,408. The cutting jig can then be rigidly fixed onto the pins by some appropriate technique such as affixing a third pin to the bone through another hole 413 whose bore is at an angle offset from the angle of the first two pins 411, 412. The third pin 413 may have a head in order to provide even better rigid affixation of the jig to the bone. The surgeon could then make the cut or cuts with a surgical saw 202 using the cutting jig as a guide. Optionally, the saw itself may also bear a marker (as shown in FIG. 2A) and be tracked by the navigation system for added safety and precision.

In accordance with the present invention, the procedure for mounting a surgical instrument such as the aforementioned tibial cutting jig is greatly simplified for the surgeon. Particularly, in accordance with the present invention, less manual dexterity and less manual precision is required.

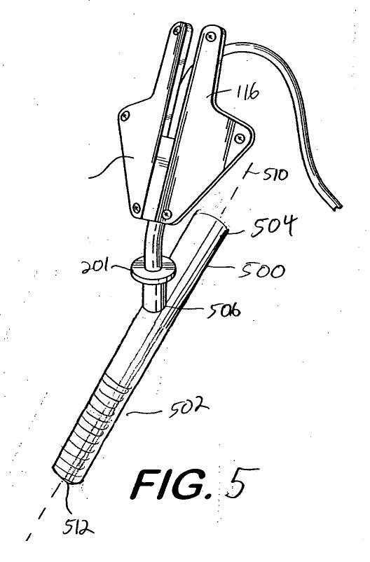

FIG. 5 is a perspective view of a navigated aiming tube 500 for use in connection with the present invention. In particular, the tube is a hollow cylinder having an inner diameter slightly larger than the pins, k-wire or other element used to mount the tibial cutting jig. The tube includes a mounting mechanism 506 for accepting the mounting mechanism 201 of one of the markers 116 used by the surgical navigation system. The surgical navigation system is preprogrammed with information defining the longitudinal axis 510 and the position of the tip 512 of the tube 500 relative to the marker 116.

Instead of navigating the cutting jig in all three aforementioned degrees of freedom, the surgeon instead first navigates and installs one of the pins upon which the cutting jig will be mounted, such as pin 411, by navigating the aiming tube 500 to define the height and axis of the hole for the pin. The position of the tip 512 of the tube when placed on the bone will define the height of the cutting jig while the orientation of the longitudinal axis 510 of the pin in the lateral plane defines the slope of the cutting jig when it is mounted on that pin. The surgeon will place pins, k-wire or other element using the navigated aiming tube 500 as a guide.

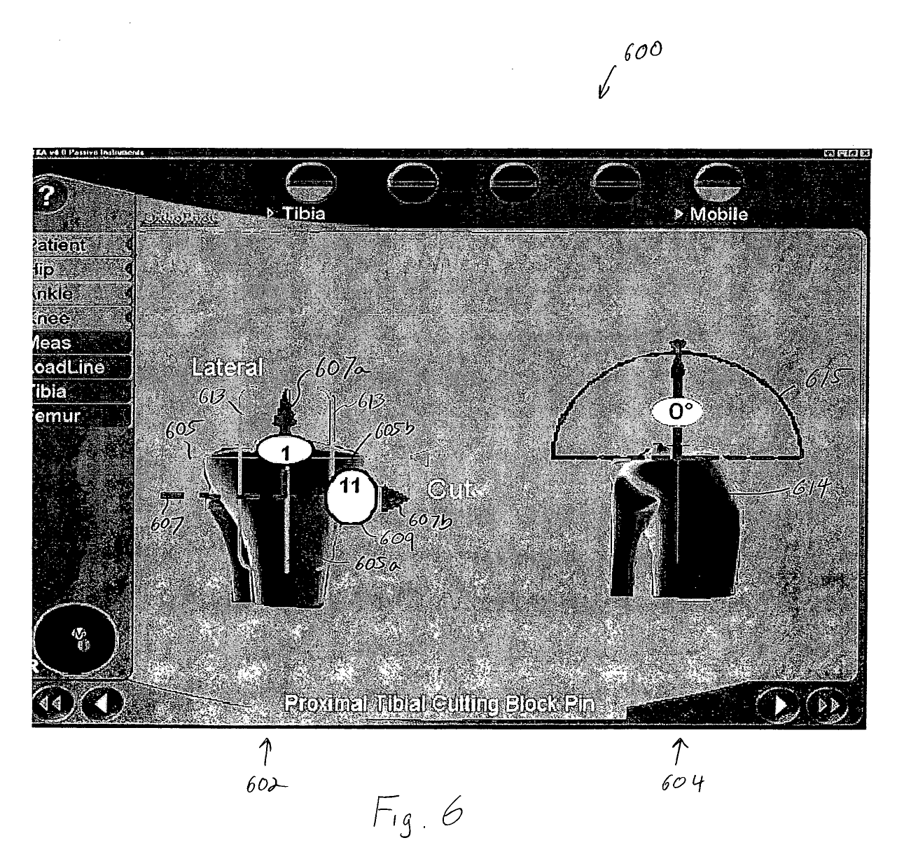

FIG. 6 is an illustration of an exemplary display screen in accordance with the present invention for navigating the tube 500. Screen 600 shows two pictorial representations of the tibia, the first 602 in the frontal plane and the second 604 in the lateral plane. The mechanical axis of the tibia has already been determined by the surgical navigation system and the tibia already bears a marker which is being tracked by the surgical navigation system, as is conventional. In the frontal view 602 on the left hand side of the display screen 600, a green cross-hair 605 (comprising horizontal line 605a and vertical line 605b) represents the mechanical axis of the tibia and a red partial cross-hair 607, comprising a vertical arrowed line 607a and a horizontal arrowed line 607b represents the aiming tube 500.

In the left hand, frontal view 602, the red partial cross-hair 607 represents the position of the tip of the tube in the frontal plane. Specifically, the height of horizontal line 607b represents the height in the frontal plane and the position of the vertical line 607a in the horizontal direction represents the horizontal position of the tip of the tube. Also, in the left hand, frontal view, the position information is redundantly shown numerically. Specifically, the height or vertical position of the tip of the tube is shown numerically in circle 609. As before, the number in circle 609 is the number of millimeters below the tibial plateau. Furthermore, the horizontal position of the tip of the tube is redundantly shown in circle 611 in which the number in the circle indicates the lateral or horizontal offset from the center of the tibia.

In a preferred embodiment, the position information shown actually is not the position of the tip of the tube per se, but is the position of the cutting plane of the cutting jig were it to be mounted on a pin placed in a hole in the bone having the position and axis defined by the aiming tube. In other words, the ultimate goal of the surgeon is to correctly position the cutting plane. As is apparent in FIG. 5, the height or slope of the pin that will be mounted in the hole created using the aiming tube 500 as a guide probably is offset from the height of the cutting plane of the jig (depending on the particular jig). Likewise, the slope of the pin may be offset from the slope of the cutting plane. Since it is the cutting plane that ultimately matters, it is preferable to have the navigation system directly convert the position of the aiming tube 500 to the position of the cutting plane rather than to show the position of the tube and require the surgeon to make the necessary conversion in his head. For example, if we assume that, for the cutting jig that will be used in the procedure, the cutting plane is actually 6 mm above the mounting pin used to mount the jig, then the display of FIG. 6 will show zero when the tube's tip is 6 mm below the desired cutting height (e.g., the height of the tibial plateau).

Bracket pair 613 is optionally provided to show the lateral range within which the pin may be safely placed. Particularly, while the height of the pin is important and must be very precisely placed, the lateral position of the pin is much less significant for TKA and will be adequate as long as it is within the area enclosed by the bracket. As a practical matter, since the system that is being mounted is offset from the center of the cutting jig (see, for instance, FIG. 5), one will generally want to mount the pin on one side of the center probably about 4 to 5 millimeters from the vertical center line of the tibia.

On the other hand, if the lateral distance from center is greater than about 5 mm, it could alter the height of the cut depending on the varus-valgus angle that is later navigated in the next navigation step. Therefore, navigation software can be provided with additional functionalilty to correct for this. Particularly, the software can be designed to calculate the change in the height of the cut depending on the lateral position of the cutting jig (as dictated by the lateral position of the navigated pin and the varus-valgus angle). Of course, in order to do this, the software must know the varus-valgus angle during this first navigation step before that angle is set. This can be dealt with in at least two ways. First, the software can simply assume that the varus-valgus angle is to be zero, since this will probably be the case in over 99.9% of surgeries. Alternately, the system can provide a screen ahead of time in which the surgeon inputs the desired varus-valgus angle. The software will then show in circle 609 the height of the cut factoring in the set or preset varus-valgus angle. Of course, as long as the surgeon mounts the jig within about 5 mm of the center in the frontal plane, this additional calculation will have little or no effect on the displayed cut height.

The slope of the cutting jig will be defined by the vertical angle of the pin. This angle is navigated in the right hand, lateral view 604. Particularly, the green cross hair 614 represents the mechanical axis of the tibia and the red semicircle represents the slope of the tube. Once again, in a preferred embodiment, the computer automatically converts the slope of the tube to the slope of the cutting plane and displays the slope of the cutting plane that is defined by the slope of the tube, rather than the slope of the tube itself.

The height of the tube is not represented in the lateral view, although it optionally could be represented by the height of the red semicircle or numerically in another circle. However, in the preferred embodiment illustrated in FIG. 6, the height of the red semicircle 615 in the lateral view does not change regardless of the height of the aiming tube since showing the height in the lateral view 604 would simply be redundant of the height information shown in the frontal view 602. It is believed that it is actually more visually pleasing to show information only as to the tube's tip position in only one of the views and to show only the slope in the other view.

In accordance with the invention, the surgeon can locate the tip of the tube at the proper height and within the appropriate horizontal range by observing the left hand, frontal view 602 and set the slope of the tube by observing the right hand, lateral view 604. The navigated height of the tube defines the height of the cutting plane. The slope of the tube defines the slope of the cutting plane.

Note that there is no representation of the position of the tip of the tube, along the third axis (which would be the axis in and out of the page in the frontal view or the axis running left to right in the lateral view). In alternate embodiments, the position of the tip of the pin along that axis could be represented in the right hand, lateral view 604. However, it is believed that there is no need to show that information as it is obvious that the tip of the tube will be placed against the surface of the bone and thus this is not a degree of freedom that needs to be navigated. Displaying unnecessary or irrelevant information is likely to add confusion rather than help the surgeon.

Now the surgeon can drill the pin in using the navigated aiming tube 500 as a guide. Specifically, the bit of a drill can be inserted into the tube and the drill energized to drill the appropriate hole. Then the pin can be screwed into the hole. That pin defines the slope and height of the cutting jig that will be mounted on the pin. Thus, in accordance with the invention so far, the surgeon has defined the slope and the height of the cutting plane by navigating only a single tube rather than the entire cutting jig and only in two degrees of freedom (height and slope) rather than three.

In alternative embodiments of the invention, the navigation need not be of an aiming tube. For instance, one may mount a marker to the pin directly and screw the pin in. In an even further embodiment of the invention, a marker may be mounted directly on to the drill that will be used for drilling the hole for the pin. In an even further embodiment, the pin can be premounted on the cutting jig and the block manipulated.

Note that, once the position of the tip of the aiming tube is defined (in the left hand, frontal view), the angle of the tube still has two degrees of freedom. We might call these degrees of freedom the vertical angle (which essentially defines the slope of the cutting plane as discussed above) and the horizontal angle. In the embodiment described above, there is no navigation of the horizontal angle of the pin. This is because the horizontal angle of the pin does not need to be set particularly precisely. As long as the pin is within about five degrees in either direction of perpendicular to the frontal plane, the cutting jig will mount and permit a good cut completely through the tibia without interference from other anatomical structures. However, if desired, the horizontal angle of the cutting plane can also be navigated, such as by providing the relevant information in the frontal view.

In fact, in one preferred embodiment of the invention, the cutting jig itself may be provided with a protrusion near one of the two sets of mounting holes 406, 408, such as a sharp pin that digs a bit into the bone or a semi-sphere fabricated from a high friction material, that can act as a pivot point for the jig. In this embodiment, the marker is mounted directly on the jig rather than an aiming tube and the jig is navigated directly by navigating the position of the protrusion (which dictates the height of the cutting plane) and rotating the jig around the pivot point (which dictates the slope of the cutting plane). A mounting pin can then be inserted through one of the holes in holes set 406 or 408 to fix the jig to the bone at the navigated height and slope.

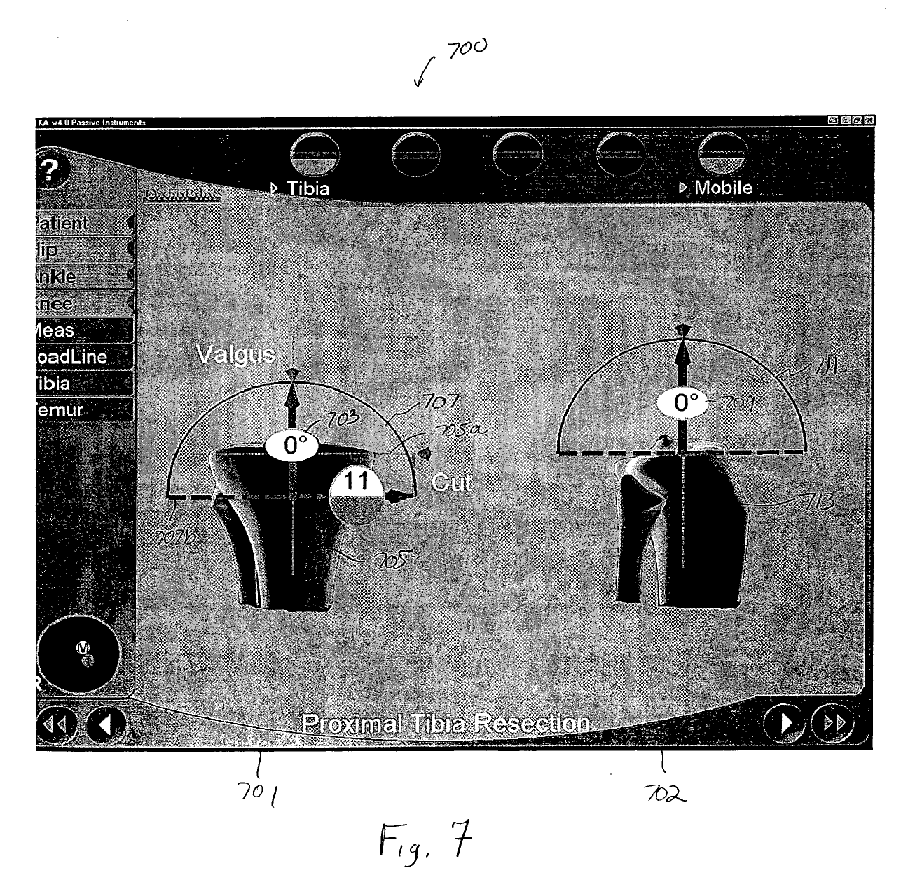

At this point, navigating the last degree of freedom, i.e., the varus-valgus angle, is simple and requires minimal manual dexterity. Particularly, after the first pin 411 or 412 is installed as described above in connection with FIG. 6, the surgeon slides the cutting jig over the first pin and then observes a screen such as screen 700 shown in FIG. 7. As in FIG. 6, the left hand view 701 is the frontal view and the right hand view 702 is the lateral view. With the cutting jig mounted over the first pin, its only relevant degree of freedom is the rotational degree of freedom about the axis of the first pin. This degree of freedom, of course, defines the varus-valgus angle of the cutting plane and is visible in the frontal view. (Technically, the jig has another degree of freedom, i.e., it can be slid along the axis of the pin, but this does not need to be navigated since, obviously, the surgeon will simply slide the jig along the pin until the jig rests against the bone.)

Therefore, only the frontal view 701 need be presented and only information as to the varus-valgus angle need be shown. However, in a preferred embodiment, the display 700 continues to show the lateral view 702 as well as the information as to height and slope for reasons that will be made clear below.

The varus-valgus angle is shown by the angle of green cross hair 705 (representing the mechanical axis of the bone) relative to the red semicircle 707 (representing the cutting plane of the cutting jig). The same information is shown redundantly numerically in circle 703. The surgeon merely needs to rotate the cutting jig about the already mounted pin until the desired varus-valgus angle is achieved. As previously noted, this angle is typically zero, i.e., the cutting plane is perfectly perpendicular to the mechanical axis of the bone. The surgeon then mounts the second pin in one of the holes in the second set of holes 408 to set the final degree of freedom (varus-valgus angle) of the cutting plane. In one embodiment of the invention, the surgeon simply inserts the pin in one of the holes in the second set of holes 408 and drills in the second pin.

The surgery can then proceed in the conventional fashion. For instance, typically the next step will be to mount a third pin at an offset angle from the first two pins through mounting holes 413 in the cutting jig in order to keep the cutting jig from sliding in and out off of the first two pins.

As mentioned above, in a preferred embodiment, screen 700 also shows the anterior angle of the cutting plane (see circle 709, red semicircle 711, and green cross hair 713 in the lateral view 702) and the height of the cutting plane (see circle 713 and the height of base 707a of red semicircle 707 relative to the horizontal line 705a of green cross hair 705 in the frontal view 701). This is because some cutting jigs, such as the one illustrated in FIG. 4, provide mechanisms to fine tune the slope and cutting height even after the jig has been fixedly mounted to the bone. For instance, with reference to FIG. 4, thumb gear handles 416, 417, and 418 actually operate gears that permit fine tuning of each of the three degrees of freedom. Particularly, handle 416 can be rotated to fine tune the varus-valgus angle, handle 417 can be rotated to fine tune the height of the cutting plane and handle 418 can be manipulated to fine tune the slope of the cutting plane. Thus, in the screen shown in FIG. 7, if the surgeon determines that small adjustments are necessary to any of the three degrees of freedom, such adjustments can be made without the need to remount the cutting jig.

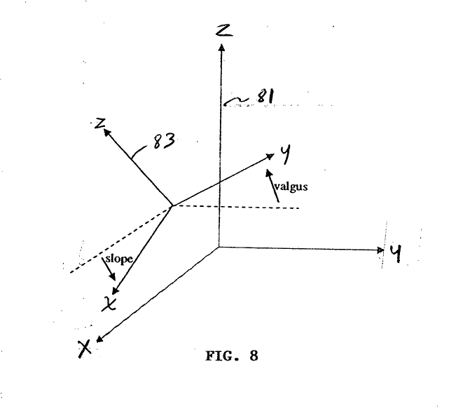

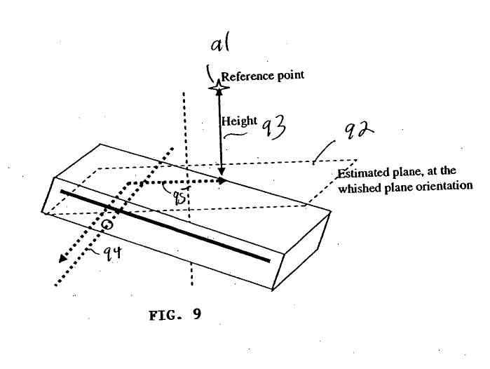

A similar process can be performed in order to navigate a femoral cutting jig. In particular, the femoral cutting jig also needs to be mounted properly in essentially the same three degrees of freedom and may be mounted in a similar manner using two mounting pins (and possibly a third, offset mounting pin). The procedure would be so similar to that described for mounting the tibial cutting jig, that we describe herein only the relevant screen views. FIG. 8 illustrates an exemplary screen 800 including a frontal view 801 (i.e., looking at the medial-lateral plane) and a lateral view 802 (i.e., looking at the sagittal plane) that would be the first screen used in navigating the height and slope of the femoral cutting jig. FIG. 9 illustrates the second screen 900 for navigating the third degree of freedom, the varus-valgus angle.

In the frontal view 801, the green brackets 805 indicate the lateral range within which the first pin should be placed and the green cross hair 807 comprising vertical line 807a and horizontal line 807b represents the mechanical axis of the femur with the height of horizontal line 807b representing the height of the surface of the distal condyles. The red partial cross hair 809 comprising vertical line 809a and horizontal line 809b represents the position of the cutting jig. Specifically, its height is represented by the height of horizontal line 809b and its lateral position represented the lateral position of vertical line 809a, just as in FIG. 6 for the tibial cutting jig. The number in circle 814 redundantly represents the height of the cutting plane or pin or aiming tube relative to the surface of the distal condyles and the number in circle 815 represents the lateral distance from the center of the femur of the tube or pin. In the right hand, lateral view 802, the orientation of the red semicircle 817 (representing the jig) relative to the green cross hair 819 (representing the mechanical axis of the femur) as well as the number in circle 816 represents the angular offset between the cutting jig and the mechanical axis of the femur.

In the second navigation screen 900, shown in FIG. 9, in the frontal view 901, the red semicircle 903 and the green cross hair 905 represent, respectively, the femoral cutting jig and the mechanical axis of the femur. The number in circle 909 represents the varus-valgus angle of the cutting jig relative to the mechanical axis. The number in circle 911 represents the height of the cutting plane above the left epicondyle and the number in circle 913 represents the height of the cutting plane above the right epicondyle. As was the case with respect to the tibia, the lateral view 902 is provided showing the slope of the cutting jig numerically in circle 915 and graphically by the relative orientation of red semicircle 917 (representing the jig) to green cross hair 919 (representing the mechanical axis of the femur) even though they have already been set in connection with the first screen of FIG. 8. As above, the jig preferably permits fine tuning of all three degrees of freedom even after the jig is fixedly mounted to the femur.

For exemplary purposes, the following is a brief discussion of one technique for calculating the height and anterior slope that will be displayed in the screen illustrated by FIG. 6 for setting the height and anterior slope of the cutting plane of the tibial cutting jig (by navigating an aiming tube, pin, drill, or the cutting jig itself). The calculations for determining the varus-valgus angle of the cutting plane that will be displayed in the screen illustrated by FIG. 7, for instance, once the height and anterior slope are set should be apparent and, therefore, will not be described herein.

The technique described below is particularly elegant as it accounts for the lateral distance from the center is illustrated in connection with FIGS. 8, 9 and 10. However, as mentioned above, simpler calculations may be implemented if one is willing to assume that the aiming tube, pin, drill, or cutting jig always will be mounted within a reasonable distance from the center of the bone (such that such distance will have a negligible impact on the height calculation).

FIG. 8 illustrates the varus-valgus angle and the anterior slope relative to the mechanical axis of the bone. Particularly, the z axis of coordinate system 81 represents the previously determined mechanical axis of the bone. The plane defined by the x and y axes would, therefore, represent the desired cutting plane, assuming that the desired cutting plane is perpendicular to the mechanical axis of the bone, as would be the case in the vast majority of procedures. In this illustration, the xz plane is the sagittal plane while the yz plane is the medial-lateral plane.

In any procedure in which the desired cutting plane is not perpendicular to the mechanical axis of the bone, the coordinate system 81 would simply be adjusted accordingly such that the xy plane of coordinate system 81 is parallel to the desired cutting plane.

Coordinate system 83 represents the cutting jig, in which the xy plane of coordinate system 83 represents the cutting plane and the z axis represents the axis perpendicular to the cutting plane. The varus-valgus angle, therefore, is the angular difference between the y axis of the cutting plane coordinate system 83 projected into the yz plane of the mechanical axis coordinate system 81, on the one hand, and the y axis of coordinate system 81, on the other hand. In this specification, a reference to projecting or projection of a line (or vector) into a plane means moving that line into that plane such that every point on that line is moved only in the direction perpendicular to that plane. In more visual terms, it is the shadow that would be cast by the actual line or vector onto the plane by a light source the light rays of which were all perpendicular to that plane.

The anterior slope of the cutting plane is the angular difference between the x axis of the cutting plane coordinate system 83 projected into the xz plane of the mechanical axis coordinate system 81 and the x axis of coordinate system 81.

FIG. 9 illustrates the calculations associated with accurately determining the height of the cutting plane of the jig relative to a reference point without the need for any assumptions as to the position of the cutting jig. While any reference point may be used for height, in one embodiment of the invention, the reference point is the point in the center of the tibial plateau. The height is herein defined as the distance measured perpendicular to the desired cutting plane (i.e., in most instances, in the direction of the mechanical axis of the bone) between the reference point and what we will call the estimated cutting plane 92. The estimated cutting plane is defined herein as the plane that intersects the vector 95 parallel to the desired cutting plane orientation and the vector 94 parallel to the varus-valgus plane and lying in the cutting plane of the cutting jig. Of course, vector 94 must be converted from the orientation of the aiming tube, drill, pin, or jig, which is easily done.

By calculating the height of an estimated cutting plane in this manner, the height number that will be displayed in FIG. 6 will accurately represent the height that the cutting plane will be when the jig is properly oriented in anterior slope and varus-valgus angle regardless of whether the cutting plane actually is oriented with the desired anterior slope or varus-valgus angle at any given instant.

In this manner, the determination of the height is completely independent of any rotation of the jig around the axis defined by the mounting pin, aiming tube, drill or cutting jig.

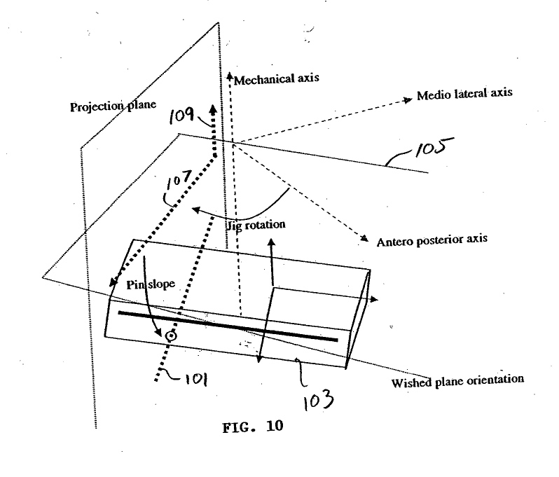

FIG. 10 is a diagram illustrating the technique for calculating the anterior slope that will be displayed in FIG. 6. The anterior slope of the cutting plane relative to the slope or angle of the aiming tube, drill, pin or jig is known. For instance, let us assume that the device being navigated is the pin, aiming tube, or drill (all of which have an easily definable longitudinal axis), instead of the jig itself. (This assumption is made for purposes of simplifying the discussion, but it will be understood that the jig itself also may be navigated exactly as described hereinbelow). The anterior slope of the longitudinal axis of the aiming tube, drill, or pin is converted into the anterior slope of the actual cutting plane by projecting its longitudinal axis into the actual cutting plane 103 of the jig. This axis is shown as axis 101 in FIG. 10. The anterior slope displayed in the screen illustrated in FIG. 6, therefore, is calculated as the difference between the aforementioned projected longitudinal axis 101 and the further projection of that axis projected onto the desired cutting plane 105. This vector is shown as vector 107 in FIG. 10. The direction of the projection is illustrated by vector 109.

A virtually identical set of calculations can be employed to navigate a femoral cutting jig in the same three degrees of freedom.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A method of using a surgical navigation system for positioning a medical device relative to an anatomical feature, said method comprising the steps of:
(1) navigating the medical device relative to the anatomical feature in a first degree of freedom;
(2) subsequently fixing the medical device to the anatomical feature in said first degree of freedom; and
(3) subsequently navigating the medical device relative to the anatomical feature in a second degree of freedom.

2. The method of claim 1 further comprising the step of:
(4) subsequently to step (3), fixing said medical device to the anatomical feature in said second degree of freedom.

3. The method of claim 2 wherein step (4) comprises fixing said medical device in multiple degrees of freedom.

4. The method of claim 2 wherein:
step (1) comprises navigating said medical device in multiple degrees of freedom; and
step (2) comprises fixing said medical device in said multiple degrees of freedom.

5. The method of claim 4 wherein said anatomical feature comprises a bone and said medical device comprises a cutting jig for cutting said bone, said cutting jig mountable to said bone by at least first and second mounting devices, said first mounting device defining said first degree of freedom and said second mounting device relative to said first mounting device defining said second degree of freedom and wherein step (1) comprises navigating said first mounting device relative to said bone and step (2) comprises navigating said second mounting device relative to said bone.

6. The method of claim 5 wherein step (4) comprises mounting said cutting jig to said bone using said first and second mounting devices.

7. A method of using a surgical navigation system for navigating a cutting jig for cutting a bone relative to said bone in multiple degrees of freedom, said cutting jig being mountable to said bone by first and second pins, said method comprising the steps of:
(1) navigating the first pin relative to the bone in at least a first degree of freedom that will define said first degree of freedom of said jig;
(2) mounting said first pin to said bone;
(3) mounting the jig on the first pin; and
(4) navigating the jig relative to the bone in at least a second degree of freedom while mounted on said first pin.

8. The method of claim 7 further comprising the step of:
(5) subsequently to step (4), mounting said second pin to said bone using said jig as a guide.

9. The method of claim 8 wherein step (1) comprises:
(1.1) mounting a marker that said surgical navigation system can track on a tube;
(1.2) navigating said tube in said at least first degree of freedom; and
(1.3) fixing said first pin in said bone using said tube as a drill guide.

10. The method of claim 8 wherein step (1) comprises:
(1.4) mounting a marker that said surgical navigation system can track on a drill;
(1.5) navigating said drill in said at least first degree of freedom; and
(1.6) drilling a hole for said first pin in said bone with said drill.

11. The method of claim 8 wherein step (1) comprises:
(1.7) mounting a marker that said surgical navigation system can track on said pin; and
(1.8) navigating said first pin in said at least first degree of freedom.

12. The method of claim 8 wherein step (1) comprises the steps of:
(1.9) displaying on a monitor in real time the position of a medical device relative to said bone in at least two degrees of freedom.

13. The method of claim 12 wherein said cutting jig is a tibial cutting jig and said bone is a tibia and wherein, in step (1.7), one of said two degrees of freedom is height of a cutting plane of said jig relative to said tibia and the other of said two degrees of freedom is anterior angle of said cutting plane relative to said tibia.

14. The method of claim 13 wherein said height is displayed in a frontal plane view and said slope angle is displayed in a lateral plane view.

15. The method of claim 14 wherein said second degree of freedom is varus-valgus angle.

16. The method of claim 15 wherein said varus-valgus angle is displayed in a frontal plane view.

17. A computer readable product embodied on computer readable media readable by a computing device for generating a display for a surgical navigation system to be used for positioning a medical device relative to an anatomical feature, the product comprising:
first computer executable instructions for generating a first graphical user interface that illustrates the relative position of the medical device relative to the anatomical feature in a first degree of freedom, whereby a surgeon can position said medical device relative to said anatomical feature in said first degree of freedom;
second computer executable instructions for switching the display to a second graphical user interface responsive to a command; and
third computer executable instructions for generating said second graphical user interface that illustrates the relative position of the medical device relative to the anatomical feature in a second degree of freedom, whereby a surgeon can position said medical device relative to said anatomical feature in said second degree of freedom.

18. The computer readable product of claim 17 wherein said first graphical user interface illustrates the relative position of the medical device relative to the anatomical feature in two degrees of freedom, whereby a surgeon can position said medical device relative to said anatomical feature in two degrees of freedom.

19. The computer readable product of claim 17 wherein said anatomical feature comprises a bone and said medical device comprises a cutting jig having a cutting plane for guiding a tool for cutting said bone, said cutting jig mountable to said bone by at least first and second mounting devices, said first mounting device defining said first degree of freedom and said second mounting device relative to said first mounting device defining said second degree of freedom and said first graphical user interface comprises a display that illustrate the position of said cutting plane in said two degrees of freedom and said second graphical user interface comprises a display that illustrate the position of said cutting plane in said second degree of freedom.

20. The computer readable product of claim 17 wherein said anatomical feature comprises a bone and said medical device is mounted to said bone via first and second mounting devices and wherein a position of said first mounting device dictates a position of said medical device in at least said first degree of freedom and a position of said second mounting device dictates a position of said medical device in at least said second degree of freedom, wherein said first computer executable instructions are adapted to navigate said first mounting device relative to said bone and said third computer executable instructions are adapted to navigate said second mounting device relative to said bone.

21. The computer readable product of claim 20 wherein said first computer executable instructions are adapted to navigate said first mounting device relative to said bone by navigating a guide device for guiding a position of said first mounting device.

22. The computer readable product of claim 20 wherein said guide device is a drill.

23. The computer readable product of claim 20 wherein said guide device is a tube.

24. The computer readable product of claim 17 wherein said medical device is a tibial cutting jig and said bone is a tibia and wherein said two degrees of freedom comprising said first degree of freedom comprises a height of a cutting plane of said jig relative to said tibia and an anterior angle of said cutting plane relative to said tibia.

25. The computer readable product of claim 23 wherein said first computer readable instructions display said height in a frontal plane view and said slope angle in a lateral plane view.

26. The computer readable product of claim 24 wherein said second degree of freedom is varus-valgus angle of said cutting plane.

27. The computer readable product of claim 25 wherein said third computer readable instructions display said varus-valgus angle in a frontal plane view.
